(19) United States
(12) Patent Application Publication
(10) Pub. No.: US 2025/0288829 A1
(43) Pub. Date: Sep. 18, 2025

(54) FAIL-SAFE VACUUM CUSHION FOR FIXING PATIENT POSITION

(71) Applicant: NATIONAL CANCER CENTER, Goyang-si (KR)

(72) Inventors: Sun Young KIM, Seoul (KR); Dong Ho SHIN, Suwon-si (KR)

(73) Assignee: NATIONAL CANCER CENTER, Goyang-si (KR)

(21) Appl. No.: 18/860,276

(22) PCT Filed: Apr. 28, 2023
(86) PCT No.: PCT/KR2023/005853

§ 371 Date: Oct. 25, 2024

(30) Foreign Application Priority Data
May 17, 2022 (KR) 10-2022-0060040

(51) Int. Cl. A61N 5/10

(52) U.S. Cl. CPC A61N 5/1077; A61N 2005/1087; A61N 2005/1097

(57) ABSTRACT

A vacuum cushion for fixing a patient's position. The vacuum cushion includes a first vacuum bag which forms a first space and is deformed according to a vacuum state and includes first beads accommodated in the first space. The vacuum cushion also has a second vacuum bag which accommodates the first vacuum bag, forms a second space together with the outer surface of the first vacuum bag, and is deformed according to the vacuum state. Second beads are also included and are accommodated in the second space. Nozzles are connected to the first space and the second space.

### TECHNICAL FIELD

The present disclosure relates to a fail-safe vacuum cushion having an immobilizer function that acts as a medical positioner, and more specifically, to a fail-safe positioner that prevents the breakage of an existing single-sided vacuum cushion used to fix a patient in a fixed position on a treatment table according to a treatment plan during radiation and proton therapy, thereby reducing a waste rate.

### BACKGROUND ART

In clinical practice, a vacuum cushion positioner is used on an examination table configured to allow a patient to assume a stable position not only in radiation oncology but also in an operating room, surgery, an endoscopy center, radiology, rehabilitation medicine, emergency medicine (ambulance and firefighting), and a veterinary hospital (laboratory), and restricts a patient's will and maintains a stable position, thereby enhancing the accuracy of treatment.

In order to fix the position of a tumor and a patient with physical discomfort during radiation therapy, a vacuum cushion positioner is required to maintain the position for a certain period of time. The vacuum cushion includes a pad filled with air and a bead provided in the pad to fix the patient's body in a vacuum state.

The cushion may be provided in various sizes, ranging from small to large, without any limitations on size and shape, and is provided with a single nozzle on an outer side thereof to supply or discharge air.

The patient may lie on the pad in a free position or parts of the body may be supported thereon. When the air is released from the cushion, a tube contracts and the bead contained in the cushion performs the function of providing a desired shape.

After performing the function of fixing the patient's custom body shape, the patient's body shape is maintained even when separated, and the patient may assume the same position even when receiving periodic radiation therapy.

The vacuum cushion is characterized by a vacuum bag made of urethane resin capable of maintaining vacuum, and 1 to 2 mm Styrofoam particles contained in the bag.

However, the conventional vacuum cushion has the following problems.

If the vacuum cushion has a single side during radiation and proton therapy, a damage rate increases. If the cushion is damaged, there is a problem that a patient undergoing treatment has to start an entire treatment process again from the beginning.

Since the positioner should be re-positioned to a state before it is damaged and Re-CT scanning and Re-Plan (radiation treatment planning) processes should be performed again, the patient suffers additional radiation exposure and is unable to receive continuous treatment for approximately 5 days. Further, additional imaging and treatment planning costs may not be charged to the patient, resulting in losses.

To solve this problem, a double pad is configured, so that even if an outer pad is damaged, an inner pad maintains its shape, thereby maintaining the continuity of radiation therapy and mitigating patient inconvenience.

### DETAILED DESCRIPTION OF INVENTION

### Technical Problems

Accordingly, the present disclosure has been made keeping in mind the above problems occurring in the related art, and an objective of the present disclosure is to provide a vacuum cushion for fixing a patient position with a double vacuum pad, which solves inconvenience caused when an existing cushion with a single-sided vacuum pad is damaged and reduces additional problems.

Further, a plurality of connecting passages that connect a space in a first vacuum bag and a space in a second vacuum bag are formed, so that the second vacuum bag is made according to the shape of the first vacuum bag when it fixes a patient, thereby solving a difficulty when each vacuum bag is made and shortening time to perform a fixing operation.

### Technical Solution

The present disclosure provides a vacuum cushion for fixing a patient's position, including a first vacuum bag forming a first space and deformed according to a vacuum state; a first bead accommodated in the first space; a second vacuum bag accommodating the first vacuum bag, forming a second space together with an outer surface of the first vacuum bag, and deformed according to the vacuum state; a second bead accommodated in the second space; and a nozzle connected to the first space and the second space.

The vacuum cushion may further include a connector connecting an outer surface of the first vacuum bag and an inner surface of the second vacuum bag.

A plurality of connectors may be provided and formed to be spaced apart from each other.

The connector may divide the second space into a plurality of sub-spaces separated from each other, and a connecting passage may be formed in the connector to make adjacent sub-spaces communicate with each other.

The first vacuum bag may have an elongated flat shape, and the connector may be formed along a side surface of the first vacuum bag.

An amount of the first bead may be 8 to 12 times an amount of the second bead.

A volume of the first space may be 8 to 12 times a volume of the second space.

A material of the first vacuum bag may have better toughness or strength than a material of the second vacuum bag.

The second vacuum bag may be made of thermoplastic polyurethane.

### Effect of Invention

According to the present disclosure, the following effects can be expected.

First, the use of a double-sided vacuum pad can dramatically reduce a waste rate due to damage caused by the use of an existing single-sided vacuum pad.

Second, reduced waste can reduce the occurrence of patient radiographic re-examinations and treatment planning.

Third, side surfaces of a first vacuum bag and a second vacuum bag of a vacuum cushion are coupled, and a plurality of connecting passages for upper and lower sub-spaces are formed, thereby preventing the two vacuum bags from being separated from each other and shortening a manufacturing time.

### BRIEF DESCRIPTION OF THE DRAWING

FIG. 1 is a perspective view illustrating a vacuum cushion according to a first embodiment of the present disclosure.

FIG. 2 is a sectional view taken along line II-II′ of FIG. 1.

FIG. 3 is a sectional view illustrating a vacuum cushion according to a second embodiment of the present disclosure.

FIG. 4 shows a first vacuum bag and a connector in the vacuum cushion according to the second embodiment of the present disclosure.

FIG. 5 is a sectional view illustrating a vacuum cushion according to a third embodiment of the present disclosure.

FIG. 6 shows a first vacuum bag and a connector in the vacuum cushion according to the third embodiment of the present disclosure.

FIG. 7 is an enlarged view of part “A” of FIG. 5.

FIG. 8 is a schematic view showing the vacuum cushion according to the third embodiment of the present disclosure in another way.

### BEST MODE FOR CARRYING OUT THE INVENTION

Hereinafter, the present disclosure will be described in detail with reference to the accompanying drawings. Since the accompanying drawings are merely examples to explain the technical idea of the present disclosure in detail, the idea of the present disclosure is not limited to the accompanying drawings.

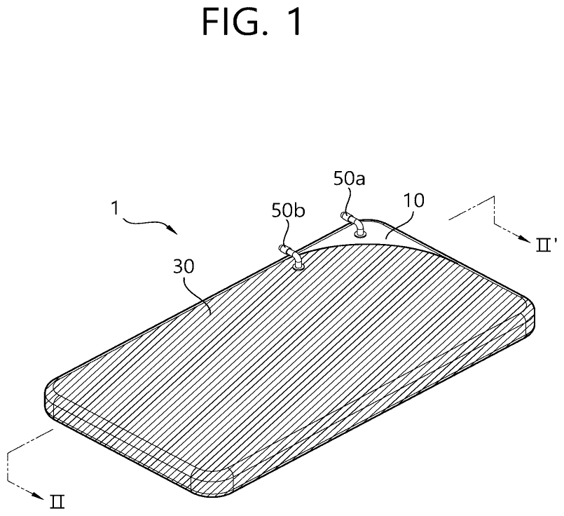

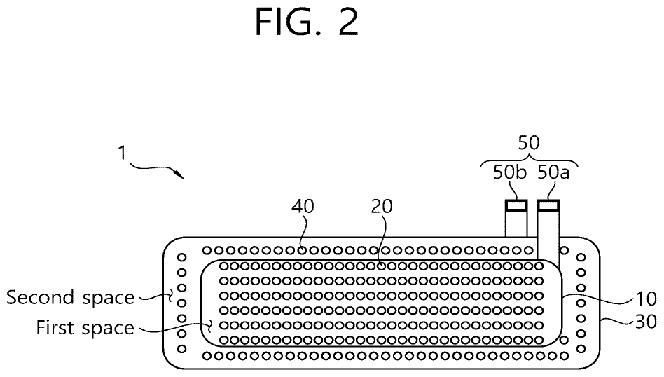

The present disclosure relates to a vacuum cushion for treating a patient, and a vacuum cushion 1 will be described with reference to FIGS. 1 and 2.

FIG. 1 is a perspective view illustrating a vacuum cushion according to a first embodiment of the present disclosure, and FIG. 2 is a sectional view taken along line II-II′ of FIG. 1. For convenience of explanation, FIG. 2 shows a nozzle 50 on a sectional view.

The vacuum cushion 1 may be of an elongated flat shape, as shown in FIG. 1.

The vacuum cushion 1 may be used as a pad or a pillow, and may have various sizes and shapes to fit for a patient's body and a patient's treatment site.

As shown in FIG. 2, the vacuum cushion 1 includes a first vacuum bag 10, first beads 20, a second vacuum bag 30, second beads 40, and a nozzle 50.

The first vacuum bag 10 forms a first space, and is deformed according to a vacuum state.

The first vacuum bag 10 is of an elongated flat shape. However, the shape of the first vacuum bag 10 is not limited thereto.

The material of the first vacuum bag 10 will be described in detail together with the material of the second vacuum bag 30 described later.

The first beads 20 are accommodated in the first space, and each have a granular shape with an average diameter of 1.5 mm to 2.0 mm.

The material of the first bead 20 may be polyethylene or polystyrene.

The first bead 20 is freely movable in the first space, and the position of the first bead 20 is fixed when the first space becomes a vacuum.

The first bead 20 has a small diameter and is solid, thereby achieving high reproducibility for the patient's fixed position.

The second vacuum bag 30 accommodates the first vacuum bag 10, and is deformed according to the vacuum state.

The inner surface of the second vacuum bag 30 forms a second space together with the outer surface of the first vacuum bag 10.

The second vacuum bag 30 is of an elongated flat shape. However, the shape of the second vacuum bag 30 is not limited thereto.

In the first embodiment, the first vacuum bag 10 and the second vacuum bag 30 are different from each other only in size, but have the same overall shape. In another embodiment, the shape of the first vacuum bag 10 may be different from that of the second vacuum bag 30.

The second vacuum bag 30 is formed of a fine denier fabric material, making it difficult to protect the patient's skin in case of a build-up effect where a dose increases due to radiation during radiation therapy. However, the occurrence rate is minimal.

The second vacuum bag 30 may be formed of thermoplastic polyurethane (TPU) fabric.

The thermoplastic polyurethane (TPU) is a plastic produced when a polyaddition reaction occurs between diisocyanate and one or more diols.

The thermoplastic polyurethane (TPU) fabric is easily bent and deforms when heat is applied thereto, exhibits excellent impact absorption, and has high resilience, outstanding compressive strain, and superior resistance to impact, abrasion, tearing, adverse weather conditions, and hydrocarbon.

The thermoplastic polyurethane (TPU) fabric has excellent flexibility without using a plasticizer, and has a wide range of hardness and high elasticity.

Further, the thermoplastic polyurethane (TPU) of the second vacuum bag 30 provides an anti-slip effect to prevent a patient from slipping when seated on the vacuum cushion 1 and also has a heat reserving effect to maintain the patient's body temperature.

The first vacuum bag 10 is made of a material with tough properties so as not to be easily damaged by impact, pressure, or friction, and has superior material strength compared to the material of the second vacuum bag 30. Therefore, even when the second vacuum bag 30 is damaged, the first vacuum bag 10 is not easily damaged, thereby maintaining the effect of fixing the patient's position.

In another embodiment, the material of the first vacuum bag 10 may be the same thermoplastic polyurethane as that of the second vacuum bag 30.

The second beads 40 are accommodated in the second space, and each have a granular shape with an average diameter of 1.5 mm to 2.0 mm.

The material of the second bead 40 may be polyethylene or polystyrene.

The second bead 40 is freely movable in the second space, and the position of the second bead 40 is fixed when the second space becomes a vacuum.

The amount of the first beads 20 may be 8 to 12 times (mass ratio) the amount of the second beads 40, and specifically, the amount of the second beads 40 may be about 10% (mass ratio) of the first beads 20.

Since the first beads 20 are provided in a larger amount than the second beads 40, the first beads 20 are provided in a sufficient amount to fix the patient's position, thereby maintaining the shape of the vacuum cushion 1, even if the second vacuum bag 30 is damaged during patient treatment and the second beads 40 do not fix the patient's position.

The volume of the first space is 8 to 12 times the volume of the second space. Specifically, the volume of the second space may be about 10% of the volume of the first space. Here, the volume refers to a volume when each space is filled with a maximum amount of air.

The nozzle 50 includes a first nozzle 50a connected to the first space, and a second nozzle 50b connected to the second space.

The nozzle 50 includes a mesh to prevent the first and second beads 20 and 40 from coming out of the vacuum bags, and may be connected to an external compressor, a suction device, a pump, etc. to change the vacuum state of the first vacuum bag 10 and the second vacuum bag 30.

The patient assumes a position on the vacuum cushion 1, and the first and second beads 20 and 40 are moved to a position where the patient's position may be fixed.

If it is determined that the fixed patient's body is in a position suitable for treatment, the second nozzle should not interfere with the first space and the second space forming the position when air discharge using the second nozzle 50b is performed 50 to 60% first and then air discharge is performed through the first nozzle 50a. Next, by discharging air using the second nozzle 50b, the vacuum state of the first vacuum bag 10 and the second vacuum bag 30 is made identical to that of the first nozzle.

At this time, air may be simultaneously discharged from the first space and the second space. In another embodiment, air may be discharged first from either the first space or the second space.

If air is discharged at the same speed at the same time, the volume of the second vacuum bag 30 is smaller than that of the first vacuum bag 10, so the second vacuum bag 30 may be in a vacuum state first. In order to solve a problem that the position fixing shape of the second vacuum bag 30 is created first while the position fixing shape of the first vacuum bag 10 is not created, a worker should control the air discharge of the first and second spaces.

The air discharge speed in the second space is adjusted so as not to interfere with the fixation of the first bead 20 in the first space.

The vacuum cushion 1 with the patient's position fixed has a low possibility of damage even if the outer second vacuum bag 30 is damaged and the inner first vacuum bag 10 is exposed to the outside because the first vacuum bag 10 is made of a tough material, so it may prevent the patient's treatment from being interrupted.

Further, since the first space in which a sufficient amount of first beads 20 are secured to fix the patient's position maintains a vacuum state as before even if the second vacuum bag 30 is damaged, the first space may be used until the end of treatment, thereby minimizing damage.

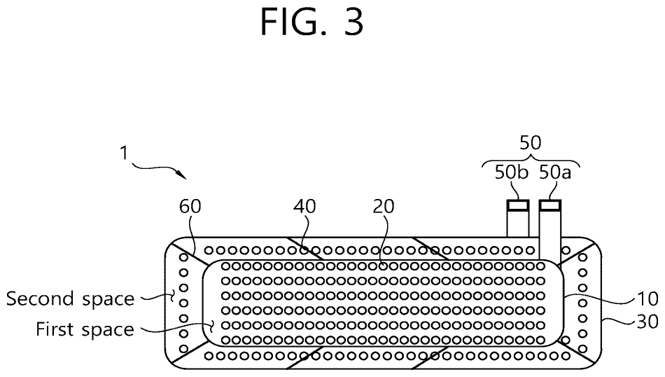

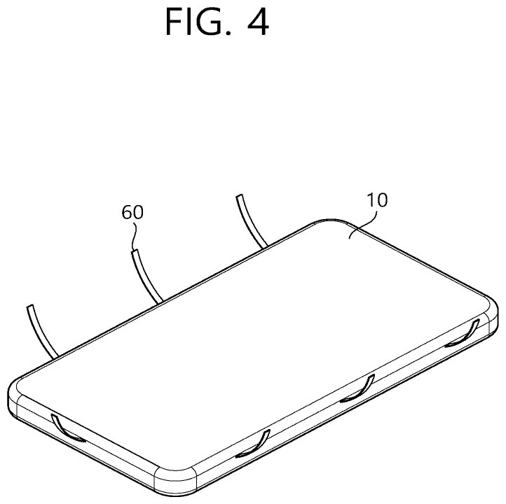

Hereinafter, a second embodiment of the present disclosure will be described with reference to FIGS. 3 and 4.

FIG. 3 is a sectional view illustrating a vacuum cushion according to a second embodiment of the present disclosure, and FIG. 4 shows a first vacuum bag and a connector in the vacuum cushion according to the second embodiment of the present disclosure.

According to the second embodiment, the vacuum cushion 1 further includes a connector 60.

The connector 60 connects the outer surface of the first vacuum bag 10 and the inner surface of the second vacuum bag 30.

As shown in FIG. 3, in the second embodiment, a plurality of connectors 60 spaced apart from each other connect the first vacuum bag 10 and the second vacuum bag 30.

As shown in FIG. 4, the connector 60 has the shape of a flat band connected to the first vacuum bag 10, and the other end of the connector 60 is connected to the second vacuum bag 30 as shown in FIG. 3.

In another embodiment, the connector 60 may have a linear shape, or may be used in a combination of linear and band shapes.

The connector 60 may solve the problems where the beads are focused on a side when the first vacuum bag 10 and the second vacuum bag 30 are in the vacuum state, and the material of the outer bag (second vacuum bag 30) is overlapped to form a shape and does not maintain a stable frame when changed to the vacuum state.

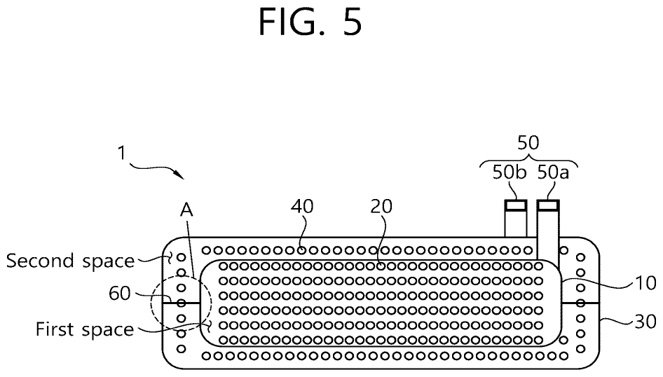

Hereinafter, a third embodiment of the present disclosure will be described with reference to FIGS. 5 to 8.

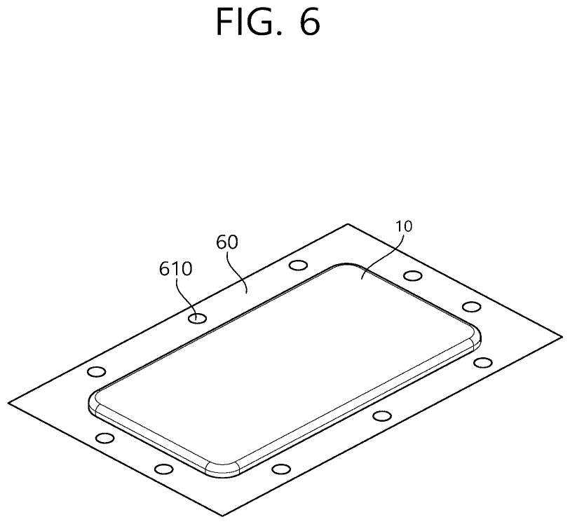

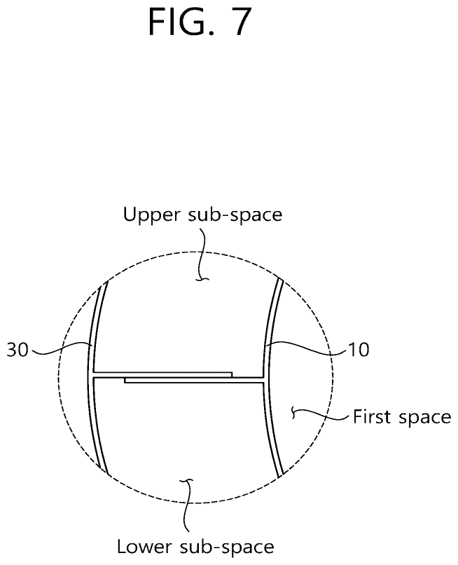

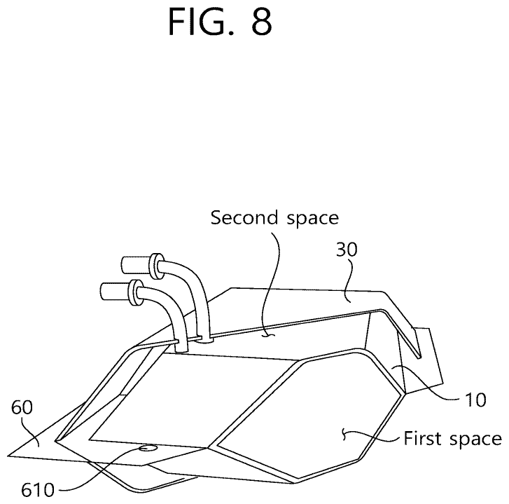

FIG. 5 is a sectional view illustrating a vacuum cushion according to a third embodiment of the present disclosure, FIG. 6 shows a first vacuum bag and a connector in the vacuum cushion according to the third embodiment of the present disclosure, FIG. 7 is an enlarged view of part “A” of FIG. 5, and FIG. 8 is a schematic view showing the vacuum cushion according to the third embodiment of the present disclosure in another way.

In the third embodiment, the vacuum cushion 1 also includes a connector 60.

As shown in FIG. 5, in the third embodiment, the connector 60 connects the first vacuum bag 10 and the second vacuum bag 30 to divide the second space into two sub-spaces, i.e. an upper sub-space and a lower sub-space.

As shown in FIG. 6, the connector 60 is formed along the side surface of the first vacuum bag 10. As shown in FIG. 5, the outer perimeter of the connector 60 is connected to the second vacuum bag 30.

The connector 60 fixes both the side surfaces of the first vacuum bag 10 and the second vacuum bag 30, thereby preventing the first vacuum bag 10 and the second vacuum bag 30 from moving separately, and providing an excellent fixing force for the first vacuum bag 10 and the second vacuum bag 30.

The connectors 60 may be extended, respectively, from the first vacuum bag 10 and the second vacuum bag 30 and coupled to each other as shown in FIG. 7. However, without being limited thereto, the connectors may be separately provided and coupled to the first vacuum bag 10 and the second vacuum bag 30, or may be extended from either of the first vacuum bag 10 and the second vacuum bag 30 and then coupled to the remaining one.

A plurality of connecting passages 610 causing sub-spaces to communicate with each other are formed in the connector 60, so a first sub-space (upper sub-space) and a second sub-space (lower sub-space) may be simultaneously changed in the vacuum state.

The connector 60 may be extended to the outside of the second vacuum bag 30, as shown in FIG. 8.

Although it is shown in FIGS. 6 and 8 that the connecting passages have circular shapes of the same size, the shape of the connecting passage 610 may be changed and the connecting passages may have different sizes.

In another embodiment, the connector 60 may divide the second space into three or more sub-spaces.

Although the present disclosure has been described above with reference to preferred embodiments, it will be apparent to those skilled in the art that specific descriptions are merely preferred embodiments and the scope of the present disclosure is not limited thereto. Therefore, the substantial scope of the present disclosure will be defined by the appended claims and equivalents thereof.

1. A vacuum cushion for fixing a patient's position, comprising:
a first vacuum bag forming a first space and deformed according to a vacuum state;
a first bead accommodated in the first space;
a second vacuum bag accommodating the first vacuum bag, forming a second space together with an outer surface of the first vacuum bag, and deformed according to the vacuum state;
a second bead accommodated in the second space; and
a nozzle connected to the first space and the second space.

2. The vacuum cushion of claim 1, further comprising:
a connector connecting an outer surface of the first vacuum bag and an inner surface of the second vacuum bag.

3. The vacuum cushion of claim 2,
wherein a plurality of connectors are provided and formed to be spaced apart from each other.

4. The vacuum cushion of claim 2,
wherein: the connector divides the second space into a plurality of sub-spaces separated from each other, and
a connecting passage is formed in the connector to make adjacent sub-spaces communicate with each other.

5. The vacuum cushion of claim 4,
wherein the first vacuum bag has an elongated flat shape, and the connector is formed along a side surface of the first vacuum bag.

6. The vacuum cushion of claim 1,
wherein an amount of the first bead is 8 to 12 times an amount of the second bead.

7. The vacuum cushion of claim 1,
wherein a volume of the first space is 8 to 12 times a volume of the second space.

8. The vacuum cushion of claim 1,
wherein a material of the first vacuum bag has better toughness or strength than a material of the second vacuum bag.

9. The vacuum cushion of claim 1,
wherein the second vacuum bag is made of thermoplastic polyurethane.
